SEQUENCE LISTING (16 sequences)

SEQ ID NO: 1 - DNA, 18 bp, Artificial Sequence
  Synthetic
cctgcttcaccaccttct

SEQ ID NO: 2 - DNA, 18 bp, Artificial Sequence
  Synthetic
atgaccacagtccatgcc

SEQ ID NO: 3 - DNA, 23 bp, Artificial Sequence
  Synthetic
ccagctacgaatctcggaccacc

SEQ ID NO: 4 - DNA, 33 bp, Artificial Sequence
  Synthetic
ttaggaagacacaaattgcatggtgaagtcagt

SEQ ID NO: 5 - DNA, 24 bp, Artificial Sequence
  Synthetic
gttgtgaggacatgtggaagcact

SEQ ID NO: 6 - DNA, 24 bp, Artificial Sequence
  Synthetic
cacagctggcaatgacaagactgg

SEQ ID NO: 7 - DNA, 18 bp, Artificial Sequence
  Synthetic
cagaactgcaggtgctgg

SEQ ID NO: 8 - DNA, 18 bp, Artificial Sequence
  Synthetic
gttctctagagatgctag

SEQ ID NO: 9 - DNA, 20 bp, Artificial Sequence
  Synthetic
ccggagaggagacttcacag

SEQ ID NO: 10 - DNA, 20 bp, Artificial Sequence
  Synthetic
ggaaattggggtaggaagga

SEQ ID NO: 11 - DNA, 20 bp, Artificial Sequence
  Synthetic
agagtttgatymtggctcag

SEQ ID NO: 12 - DNA, 22 bp, Artificial Sequence
  Synthetic
tacgghtaccttgttacgactt

SEQ ID NO: 13 - DNA, 18 bp, Artificial Sequence
  Synthetic
ggattagataccctggta

SEQ ID NO: 14 - DNA, 20 bp, Artificial Sequence
  Synthetic
ccgtcaattcmtttragttt

SEQ ID NO: 15 - DNA, 20 bp, Artificial Sequence
  Synthetic
agagtttgatcmtggctcag

SEQ ID NO: 16 - DNA, 22 bp, Artificial Sequence
  Synthetic
tacggytaccttgttacgactt

(19) United States
(12) Patent Application Publication
(10) Pub. No.: US 2022/0387524 A1
(43) Pub. Date: Dec. 8, 2022

(54) NOVEL KIMCHI-DERIVED LACTOBACILLUS FERMENTUM STRAIN WITH EXCELLENT ANTI-INFLAMMATORY ACTIVITY AND COMPOSITION INCLUDING SAME FOR PREVENTION AND TREATMENT OF INFLAMMATORY DISEASES

(71) Applicants: Total Medical Solutions Healthcare Co., Ltd., Cheongju-si Chungcheongbuk-do (KR); Korea University Research and Business Foundation, Seoul (KR)

(72) Inventors: Byung Youn CHOI, Cheongju-si Chungcheongbuk-do (KR); Sang Hoon Lee, Cheongju-si Chungcheongbuk-do (KR); Mee Ae Kim, Cheongju-si Chungcheongbuk-do (KR); Young Jae Park, Cheongju-si Chungcheongbuk-do (KR); Ha Young Kim, Cheongju-si Chungcheongbuk-do (KR); Su Ji Choi, Cheongju-si Chungcheongbuk-do (KR); Sae Hun Kim, Seoul (KR); Jae Young Kim, Seoul (KR); Gyu Wan Kim, Seoul (KR)

(21) Appl. No.: 17/354,531

(22) Filed: Jun. 22, 2021

(30) Foreign Application Priority Data
May 28, 2021 (KR) 10-2021-0068826

(51) Int. Cl. A61K 35/747; C12N 1/20; A23L 33/135

(52) U.S. Cl. CPC A61K 35/747; C12N 1/205; A23L 33/135; C12R 2001/225

(57) ABSTRACT

Disclosed are a novel Lactobacillus fermentum) E4 strain isolated from Kimchi which is a traditional Korean fermented food and a mass production method for the strain. The Lactobacillus fermentum E4 strain is verified to be a safe and useful probiotic strain through acid tolerance, bile acid tolerance, gelatin liquefaction, and urease tests. In addition, high DPPH free radical scavenging (%) associated with antioxidant activity was observed, and reduction in cytokines Iinterleukin-1β (IL-1β), Iinterleukin-8 (IL-8), Iinterleukin-6 (IL-6), and Toll-like receptor (TLR4), which are associated with an inflammatory response, was observed.

### CROSS REFERENCE TO RELATED APPLICATION

The present application claims priority to Korean Patent Application No. 10-2021-0068826, filed May 28, 2021, the entire contents of which is incorporated herein for all purposes by this reference.

### SEQUENCE LISTING

The present application contains a Sequence Listing which has been submitted electronically in ASCII format and is hereby incorporated by reference in its entirety. The ASCII copy, created on Aug. 6, 2021, is named T03-540_ST25.txt and is 3,449 bytes in size.

### BACKGROUND

### 1. Field of the Invention

The present disclosure relates to a new strain of Lactobacillus fermentum E4 (KCTC 14570BP) derived from Kimchi which is a traditional Korean fermented food, and a method for mass production of the strain. The strain may be included in a prophylactic or therapeutic composition for inflammatory diseases and metabolic disorders.

### 2. Description of the Related Art

It is known that Lactobacillus or lactic acid bacteria use sugar for their growth and produce lactic acid. Lactic acid bacteria are largely categorized into the genus of Lactobacillus, Lactococcus, Streptococcus, Leuconostoc, Weissella, Pediococcus, Bifidobacterium, and Enterococcus. Among them, the Lactobacillus genus and the streptococcus genus are most widely known. Lactic acid bacteria are known to be one of the most useful microbes available to humans and are widely used in human life for various products including fermented foods such as Kimchi, soy sauce, and sausages, medicine and medical supplies, and feed additives.

Morphologically, lactic acid bacteria are classified as coccus including Lactococcus, Pediococcus, Streptococcus, and Leuconostoc, or as rods including Lactobacillus and Bifidobacterium. The lactic acid bacteria are also known as breathable anaerobic or anaerobic bacteria. Lactic acid bacteria are known to exhibit the most active growth at 37° C., stop growing at temperatures below 4° C. or above 45° C., and die at temperatures above 60° C. In addition, it is reported that most of the lactic acid bacteria are acid resistant and that the nutritional composition required for their growth is extraordinarily complex and includes many types of amino acids and vitamins as well as saccharides.

It has been recently proven in papers and patent documents that Kimchi-derived lactic acid bacteria can be effectively used for pharmaceutical products and cosmetics. This suggests that the usefulness of lactic acid bacteria is of high technical value and that the lactic acid bacteria can be developed into health functional foods and functional cosmetics which are applications of so-called intestinal-dermatological science. In addition, additional research is required on applications of lactic acid bacteria to the human body, stability and safety in the human body, and storage stability for market distribution.

Kimchi is a traditional Korean fermented food prepared by salting vegetables such as cabbage, radish, and cucumber and adding various auxiliary materials and spices to the vegetables to create an environment in which lactic acid bacteria can grow as the dominant species so that the vegetables can be fermented. It has been reported that various auxiliary materials included in Kimchi serve as resources for the growth of lactic acid bacteria so that lactic acid bacteria with different characteristics can grow depending on the type of Kimchi. Bacteria of the genus Leuconostoc and the genus Lactobacillus are mainly found in Kimchi aged for a certain period time. It is considered that the bacteria of the genus Lactobacillus, which have high osmotic tolerance and acid tolerance, can survive during the pickling process and the acid fermentation process.

On the other hand, an inflammatory response is one of the responses of the body's immune system to physical factors such as injury, heat, and radiation, chemical factors such as poisons including strong acids, pathogenic microorganisms, and immunological stimuli such as allergies. The inflammatory response is a mechanism to restore or regenerate the damaged tissue. In the in vivo tissue regeneration mechanism, macrophages play an especially important role in regulating the inflammatory response and immune function. Macrophages activated by external antigens and stimuli secrete a large amount of growth factor, cytokine, prostaglandin E2 (PGE2), lipid mediator, and nitric oxide. Among them, PGE2 promotes the secretion of inflammatory cytokine such as interleukin-6 as well as expands blood vessels, increases the permeability of the blood vessel wall, and dispatches immunologically competent cells to the inflammation site. In addition, when microorganisms invade, macrophages release or generate reactive oxygen intermediates, hypochlorite, nitric oxide, myeloperoxidase, neutral protease, lysosomal hydroxylase, and the like that are toxic to the microorganisms. However, these molecules also directly damage the body tissue.

These inflammatory responses are classified as acute and chronic by the time of progression. Depending on the cause, site, and type of the inflammatory response, there is a risk that the inflammatory response can lead to boils, sores, oral inflammation, peritonitis, inflammatory bowel disease, gastric ulcers, cystitis, tonsillitis, conjunctivitis, etc.

Korean Patent No. 10-1960352 (registered as of Mar. 14, 2019) discloses a strain named Lactobacillus brevis SBB07 (KCCM12102P) that is derived from fermented berries. The strain has good antibacterial activity against harmful microorganisms, antibiotic activity, antioxidant activity, enzyme secretion ability, acid tolerance, bile tolerance, and heat resistance, and prebiotic substrate availability, and does not produce biogenic amines. The patent also discloses antibacterial compositions and probiotic compositions containing the strain or its culture medium as an active ingredient.

Korea Patent Application Publication No. 10-2010-0045758 (published as of May 4, 2010) discloses that a Lactobacillus pentosus PL-11 strain has physiological activities such as bile and acid tolerance, anti-inflammatory effect, and enzyme decomposition ability. Therefore, when the strain is used as probiotics for fish, it is possible to protect the fish from bacteria that are not useful for fish farming, thereby improving the survival rate of the fish as well as improving the fish growth rate and feed efficiency.

### SUMMARY

An objective of the present disclosure is to develop and provide a novel Kimchi-derived strain that has antioxidant and anti-inflammatory activities, acid tolerance, and bile tolerance and which is confirmed for its safety and functionality as probiotics through a gelatin liquefaction test and a urea test (harmful metabolite production test).

Another objective of the present disclosure is to develop and provide a mass production method for the strain.

A further objective of the present disclosure is to provide a Lactobacillus fermentum E4 strain (KCTC 14570BP).

The E4 strain (KCTC 14570BP) preferably has good antioxidant activity and anti-inflammatory activity.

The present disclosure provides an anti-inflammatory food composition including a Lactobacillus fermentum E4 strain (KCTC 14570BP), a culture medium for the strain, a concentrate of the culture medium, or a dry powder of the culture medium.

The present disclosure provides an anti-aging food composition including a Lactobacillus fermentum E4 strain (KCTC 14570BP), a culture medium for the strain, a concentrate of the culture medium, or a dry powder of the culture medium.

The present disclosure provides a pharmaceutical composition for the prevention or treatment of inflammatory diseases, the composition including a Lactobacillus fermentum E4 strain (KCTC 14570BP), a culture medium for the strain, a concentrate of the culture medium, or a dry powder of the culture medium.

The present disclosure provides a method of culturing a Lactobacillus fermentum E4 strain (KCTC 14570BP), the method being characterized in that the Lactobacillus fermentum E4 strain (KCTC 14570BP) is cultured in a medium using glucose as a carbon source. In this case, the medium may preferably contain 2.00% to 2.50% (w/v) of a carbon source, 2.80% to 3.20% (w/v) of a nitrogen source, and 0.25% to 0.29% (w/v) of inorganic salts.

The present disclosure provides a new Kimchi-derived strain, called Lactobacillus fermentum E4 (KCTC 14570BP), which is verified as a safe and useful probiotic strain through tests for acid tolerance, bile tolerance, gelatin liquefaction reaction, and harmful metabolite production. In addition, high DPPH free radical scavenging (%) associated with antioxidant activity was observed, and reduction in cytokines Iinterleukin-1β (IL-1β), Iinterleukin-8 (IL-8), Iinterleukin-(IL-6), Toll-like receptor (TLR4), which are associated with an inflammatory response, was observed.

On the other hand, in the present disclosure, experiments were performed with different medium compositions for industrial mass production of the E4 strain (KCTC 14570BP), and the optimal medium composition was developed.

### BRIEF DESCRIPTION OF THE DRAWINGS

FIGS. 1A-1C show the cytokine expression levels of ten strains selected in the present disclosure;

FIG. 2 (SEQ ID NOS: 13-16) shows the result of 16S rRNA analysis of a strain E4 selected in the present disclosure;

FIG. 3 shows culture conditions created to find the optimal growth medium for the E4 strain selected in the present disclosure;

FIG. 4 shows the results of a case where the E4 strain was cultured in various culture media; and

FIG. 5 shows the results of a case where the E4 strain was cultured in an S-C medium in which glucose is included Carbon Source 1.

### DETAILED DESCRIPTION

In the present disclosure, over 200 different strains were screened to identify organisms that are effective as probiotic strains. For the strains, the stability was examined through tests for acid tolerance, bile tolerance, gelatin liquefaction, and harmful metabolite production, and antioxidant activity which is an indicator for aging and is investigated through DPPH assay. Thus, ten strains were selected as candidates for probiotic strains by combining the test results.

To assay the anti-inflammatory activity of each of the ten selected strains, a cell line HT-29 was used to determine the levels of inflammatory cytokines IL-1 β, IL-8, TLR4, and IL-6, the primer sequences, and the Tm values. Then, the average value of the cytokine expression levels for each strain was ranked, resulting in a total of four strains E4, B, E, and F being selected as high anti-inflammatory strains.

Among them, Lactobacillus fermentum E4, a new strain isolated from Kimchi, which is a traditional Korean fermented food, was confirmed to be the most optimal strain, and as of May 18, 2021, the strain was submitted to the Korean Collection for Type Cultures (KCTC), which is a depository institution of microorganisms for patent purposes in Korea and was deposited under the accession number “KCTC 14570BP”.

The E4 strain of the present disclosure was confirmed to exhibit good antioxidant activity as described below and was thus confirmed to have improved anti-aging capability indicated by the antioxidant activity. Based on this observation, it is considered that the E4 strain of the present disclosure can be used as a raw material of an anti-aging food composition. In addition, since the E4 strain of the present disclosure has good anti-inflammatory activity, the E4 strain can be used as a food composition for alleviating inflammation or as a pharmaceutical composition for the treatment or prevention of inflammation. The food composition or the pharmaceutical composition of the present disclosure may include the E4 strain, a culture medium for the E4 strain, the concentrate of the culture medium, or a dry powder of the culture medium.

Recently, probiotics have become popular as a material for food or medicine as they have shown to improve intestinal health and to have various functions, and the E4 probiotic strain according to the present disclosure can also be used as a raw material for medicine or health-functional foods.

On the other hand, in the present disclosure, the food composition is not necessarily limited to a specific formulation. Examples of the specific formulation of the food composition include meat, grains, caffeinated beverages, general drinks, chocolate, breads, snacks, confectionery, candy, pizza, jelly, noodles, gums, dairy products, ice creams, alcoholic beverages, alcohol, vitamin complexes, and other health supplements. More preferably, the formulation may be one selected from lactic acid bacteria fermented milk, soy milk, powdered milk, yogurt, beverages, granules, and health supplements, but is not necessarily limited thereto.

On the other hand, the pharmaceutical composition of the present disclosure may further include a pharmaceutically acceptable carrier, diluent, or excipient. Examples of the available carriers, excipients, or diluents include lactose, dextrose, sucrose, sorbitol, mannitol, xylitol, erythritol, maltitol, starch, gum acacia, alginate, gelatin, calcium phosphate, calcium silicate, cellulose, methylcellulose, microcrystalline cellulose, polyvinylpyrrolidone, water, methylhydroxybenzoate, propylhydroxybenzoate, talc, magnesium stearate, and mineral oil, and at least one selected from among them may be used. In addition, when the therapeutic and prophylactic composition is a drug, the composition may further contain a filler, an anti-aggregate, a lubricant, a wetting agent, a fragrance, an emulsifier, or a preservative.

On the other hand, the pharmaceutical composition according to the present disclosure may be formulated into a desirable dosage form depending on the usage thereof and particularly formulated into a dosage form by which the active ingredient therein can be released in a fast, sustained, or delayed manner after being administered to a mammal. Specific examples of the dosage form include plasters, granules, lotions, liniments, lemonades, aromatic waters, powders, syrups, ophthalmic ointments, liquids and solutions, aerosols, extracts, elixirs, ointments, fluid extracts, emulsions, suspensions, decoctions, infusions, ophthalmic solutions, tablets, suppositories, injections, spirits, cataplasms, capsules, creams, troches, tinctures, pastes, pills, and flexible or rigid gelatin capsules. The pharmaceutical composition may be formulated into any one of the exemplary dosage forms.

On the other hand, as to the pharmaceutical composition according to the present disclosure, the unit dose may be determined depending on the medication method and the age, gender, weight, and severity of the disease of the person who takes the pharmaceutical composition. For example, given the strain of the present disclosure, at least a dose of 0.00001 to 100 mg/kg (weight) per day can be administered orally. However, the dose is only an example and may vary depending on the conditions of the person taking the pharmaceutical composition and on a doctor's prescription.

In addition, the strain of the present disclosure has been checked for the potential for commercial mass production. Experiments were conducted with different combinations of media, and it was confirmed that a medium using glucose as a carbon source showed a good growth rate. It was confirmed that a medium containing 2.00% to 2.50% (w/v) of a carbon source, 2.80% to 3.20% (w/v) of a nitrogen source, and 0.25% to 0.29% (w/v) inorganic salts was preferable. In addition, it was found that the optimum culture time and temperature were 9.5 hours and 37° C.

Hereinafter, the present disclosure will be described in more detail with reference to examples and experimental examples described below. However, the scope of the present disclosure is not limited only to the examples and experimental examples described below but covers even modifications of technical ideas equivalent thereto.

### Example 1: Evaluation of Stability and Functionality of Candidate Strains for Probiotics

### 1. Purpose of Experiment

In the present experimental example, stability and functionality of candidate strains for probiotics were evaluated.

### 2. Experiment Method

(1) Acid Tolerance Test

To investigate the in vitro gastric survival of each of the candidate strains when ingested, the tolerance of each of the strains for an acid was tested through a method in which each prepared strain was exposed to simulated gastric juice and cultured at 37° C. to measure the number of living microorganisms at 0.3-hour intervals. In this case, the simulated gastric juice was adjusted with 1N HCL so that a culture medium had a pH of 2.5, pepsin was added to the medium to the extent of 1000 unit/mL, and the culture was sterilized and was diluted with a phosphate buffer (pH 6.8) containing KH₂PO₄, Na₂HPO₄, L-cysteine, HCl, Tween80, etc. After that, the difference in the total number of living microorganisms between each of the test groups and a control group was calculated. The control group was tested in the same manner on a liquid medium containing no simulated gastric juice.

(2) Bile Tolerance Test

To impart health benefits, probiotic strains must remain alive until reaching the small intestine through the stomach and the pancreas-duodenum when the probiotic strains are ingested. Thus, a bile resistance assay was conducted to investigate the resistance of each strain to the bile acid produced during the travel to the small intestine. To create a similar environment to the actual digestive system for evaluation of bile tolerance, an MRS sterile liquid medium containing 0.3% oxgall was inoculated on a culture medium that has undergone exposure to simulated gastric juice. Then, the prepared strains were cultured at 37° C. The number of living microorganisms was measured immediately after the start of the culture and at intervals of 24 hours, and the difference in the number of living microorganisms between a control group and each of the test groups was calculated. The control group was tested in the same manner on a liquid medium containing no simulated bile acid.

(3) Gelatin Liquefaction Test

To investigate the creation of gelatinase, the candidate strains for probiotics were inoculated on gelatin nutrient media (0.3% of beef extract, 0.5% of peptone, and 12% of gelatin) and were cultured at 37° C. for 48 hours. The gelatin nutrient media were maintained at 4° C. for about 4 hours and then whether the media had solidified were checked. When the media did not solidify, the media were determined to be positive for the liquid reaction.

(4) Urease Test for Checking Creation of Harmful Metabolite Products

After streaking probiotic strains on urea agar base media, the candidate strains for probiotics were cultured at 37° C. for 48 hours. When the investigated candidate strains produced urease, urea was decomposed to increase the pH of the media, thereby causing the phenol red used as a pH indicator to change from yellow to red.

(5) DPPH Free Radical Scavenging Test for Checking Antioxidant Activity

The candidate strains for probiotics were inoculated on sterilized MRS broth in an amount of 1%, cultured at 37° C. for 18 hours, and centrifuged at 10,000 rpm at 4° C. for 5 minutes to prepare cell-free extract (CFE). 500 μL of the prepared CFE was mixed with 3.0 mL of 2,2-DiPhenyl-2-Picryl hydrazyl hydrate(DPPH) solution (5 mg/100 mL ethanol). The same amount of ethanol, MRS solution, and ascorbic acid (100 μg/mL) were used as a control group, a blank, and a comparison group, respectively. After being mixed with the DPPH solution, incubation was performed in a darkroom for 30 minutes, absorbance for 517 nm was measured, and antioxidant power was calculated in percentage (%) according to Equation 1.

$$D P P H free radical scavenging ( \% ) = \frac{Absorbance of control - Absorbance of sample}{Absorbance of control} \times 100 \qquad [ Equation 1 ]$$

### 3. Results of Experiments

Ten strains with excellent safety, stability, and functionality were selected from the candidate group based on the test results shown in Table 1.

TABLE 1

| | Acid | Bile salts | DPPH free | | |
|---|---|---|---|---|---|
| | tolerance | tolerance | radical | | |
| | (Relative | (Relative | scavenging | | |
| Strain | to 0 h %) | to 0 h %) | (%) | Urease | Gelatinase |
| A | 91 | 118 | 88.09 | | |
| E4 | 88 | 108 | 81 | — | — |
| B | 87 | 114 | 80 | — | — |
| C | 88 | 105 | 77 | — | — |
| E | 99 | 110 | 81 | — | — |
| D | 100 | 109 | 75 | — | — |
| F | 82 | 111 | 80 | — | — |
| G | 83 | 104 | 80 | — | — |
| H | 79.5996.2 | 86.38 | 80.87 | — | — |
| I | 84 | 103 | 91 | — | — |

### Example 2: Evaluation of Anti-inflammatory Activity of Selected Strains

The present experiment was intended to evaluate the anti-inflammatory function of the ten selected strains.

For the experiment, the HT-29 cell line was obtained from the Korea Culture Type Collection (KCTC, Korea) and was incubated in an atmospheric environment containing 5% CO2 at 37° C. using a RPMI 1640 medium (Gibco BRL, U.S.A.) to which 10% heat-inactivated fetal bovine serum (FBS, Gibco), penicillin G (100 IU/mL), and streptomycin (100 mg/mL) were added.

The HT-29 cells were seeded on 96 well plates in a density of 1×10⁵ cells/well and incubated for 24 hours, and then the HT-29 cells were pre-processed with heat-treated strains for 24 hours. After removing the media, the cells were treated with 1 μg/mL of lipopolysaccharide (LPS) to induce an inflammatory response, and the response continued for 24 hours. There were largely three groups: positive, negative, and strain processing. The positive group was subjected to no processing, the negative group was processed only with LPS, and the strain-processed group was processed with LPS and strains.

The media in the plate wells that underwent the LPS processing were suctioned for cDNA synthesis and RNA extraction and were processed with 1 mL of Trizol reagent (Invitrogen). Next, the cells were completely separated by a cell scrapper, followed by addition of 200 μL of chloroform, stirring, 5-minute incubation, and centrifugation conducted at 4° C. or 15 minutes at 12000 rpm. Next, the supernatant was dispensed into an ep tube, 200 μL of isopropanol was added to the tube, and incubation was performed for 10 minutes. After the incubation, the centrifugation was performed at 12000 rpm for 15 minutes at 4° C., and the supernatant was removed. In this case, pellets in the tube were washed with 75% EtOH and centrifugation was performed at 7500 rpm for 5 minutes at 4° C. to remove the supernatant. Next, air drying was performed for 5 minutes, DEPC 20 μL was dispensed, and RNA concentration was measured with a NanoDrop spectrometer. The sample was diluted so that the RNA concentration was reduced to 100 ng/μL based on the measured concentration value, and cDNA was synthesized through PCR using a cDNA kit (manufactured by Applied Biosystems). The PCR was performed at 25° C. for 10 minutes, 37° C. for 2 hours, and at 85° C. for 5 minutes.

The synthesized cDNA was diluted to a concentration of 100 ng/μL, the genetic representations of inflammatory cytokines was measured using qRT-PCR, and IL-1β, IL-8, TLR4, and IL-6 were used as biomarkers of the inflammatory response, and the used primer sequences are shown in Table 2 (SEQ ID NOS 1-10, respectively).

TABLE 2

| | | | SEQ ID | Tm |
|---|---|---|---|---|
| Gene | — | Gene Sequence | NO | (° C.) |
| GAPDH | f | 5′-CCT GCT TCA | 1 | 59.8 |
| | | CCA CCT TCT-3′ | | |
| | r | 5′-ATG ACC ACA | 2 | 59.8 |
| | | GTC CAT GCC-3′ | | |
| IL-1 | f | 5′-CCA GCT ACG AAT | 3 | 63.0 |
| | | CTC GGA CCA CC-3′ | | |
| | r | 5′-TTA GGA AGA CAC AAA | 4 | 63.0 |
| | | TTG CAT GGT GAA GTC | | |
| | | AGT-3′ | | |
| IL-8 | f | 5′-GTT GTG AGG ACA | 5 | 56.5 |
| | | TGT GGA AGC ACT-3′ | | |
| | r | 5′-CAC AGC TGG CAA | 6 | 56.5 |
| | | TGA CAA GAC TGG-3′ | | |
| TLR4 | f | 5′-CAG AAC TGC | 7 | 53.2 |
| | | AGG TGC TGG-3′ | | |
| | r | 5′-GTT CTC TAG | 8 | 53.2 |
| | | AGA TGC TAG-3′ | | |
| IL-6 | f | 5′-CCG GAG AGG | 9 | 64.2 |
| | | AGA CTT CAC AG-3′ | | |
| | | 5′-GGA AAT TGG | 10 | 64.2 |
| | | GGT AGG AAG GA-3′ | | |

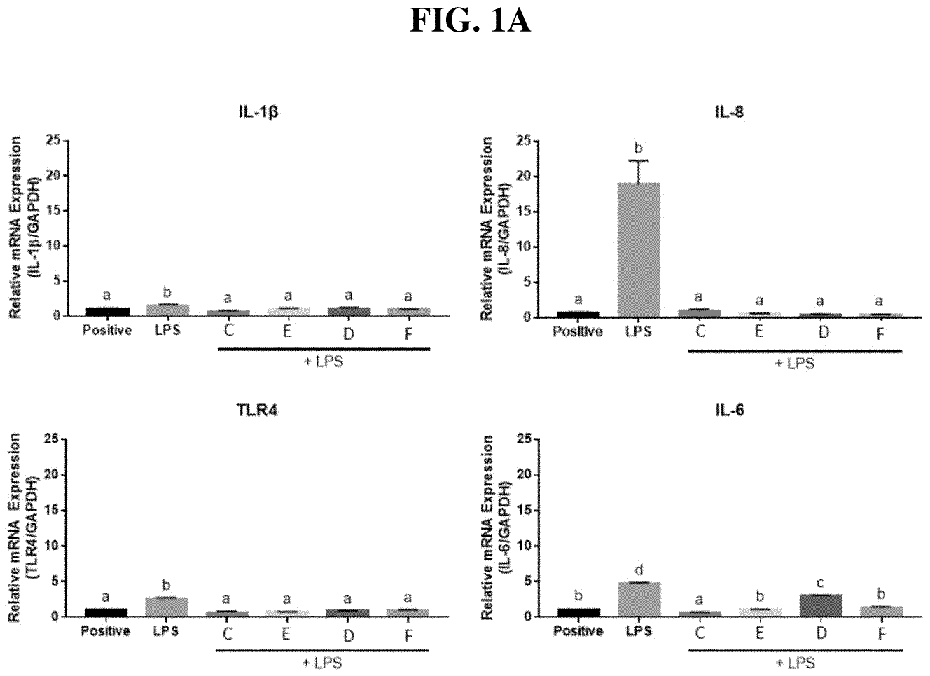

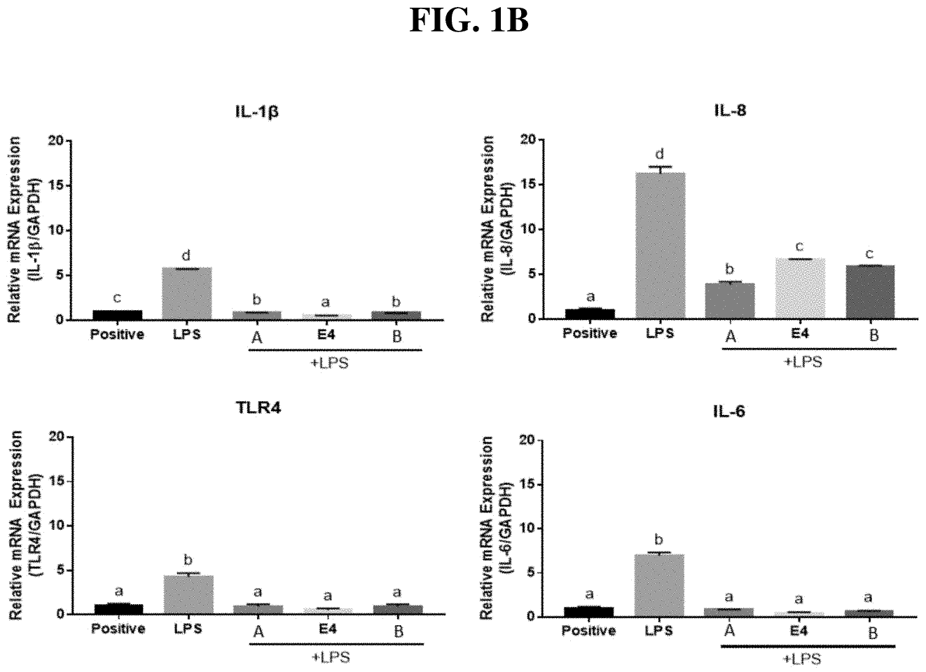

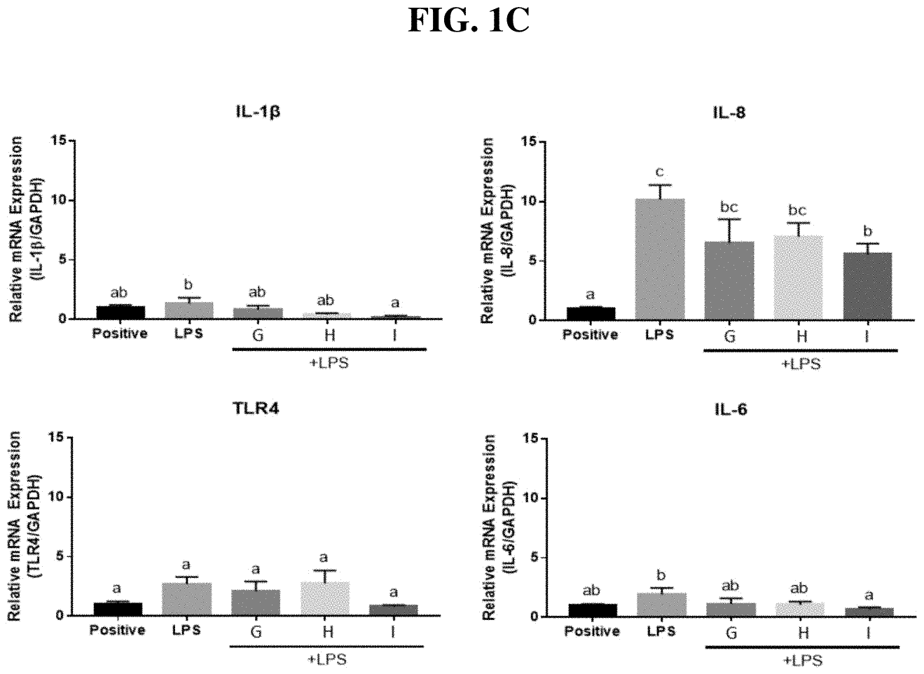

The measurement results of the expression levels of the respective inflammatory cytokines were as shown in FIGS. 1A-1C. FIGS. 1A-1C show the expression levels of the cytokines of each of the ten strains selected in the present disclosure.

The expression level measurement results for each inflammatory cytokine were collected to obtain the average expression level (%). The average expression levels were calculated using a conversion formula (Equation 2).

Expression level (%) of inflammatory cytokine=[1−(Foldₛₐₘₚₗₑ−Fold_{blank})/(Fold_{control}−Fold_{blank})]×100 [Equation 2]

Among the calculation results, only the best results for strains E4, B, E, and F are shown in Table 3.

TABLE 3

| | | Inflammatory cytokine | |
|---|---|---|---|
| | | expression inhibitory | Identification |
| Rank | Strain | effect average (%) | Result |
| 1 | E4 | 121 | L. fermentum |
| 2 | B | 110 | L. brevis |
| 3 | E | 105 | L. fermentum |
| 4 | F | 102 | L. brevis |

The cytokine expression inhibitory effects of the tested strains were found to be 121% for E4, 110% for B, 105% for E, and 102% for F. Given that the inhibitory effect of the positive group (control group) is 100%, it is determined that all the tested strains were superior to the control group. Among them, the E4 strain exhibited the highest cytokine expression inhibitory effect.

On the other hand, DNA sequencing was performed for identification of microorganisms cultured in an MRS medium, and genomic DNA was extracted from the microbial culture medium in which microorganisms were cultured, using a Genomic DNA prep kit (17121, INTRON). For the identification, the 16s rRNA gene sequence of the extracted genomic DNA was amplified with the universal primers 27F (5′-AGA GTT TGA TYM TGG CTC AG-3′) (SEQ ID NO: 11) and 1492R (5′-TAC GGH TAC CTT GTT ACG ACT T-3′) (SEQ ID NO: 12). To investigate the DNA base sequence of the PCR reaction products, the PCR reaction products were purified with a PCR purification kit (28104, Qiagen), and then used for the DNA sequencing. The DNA sequence was determined using an ABI PRISM 3700 DNA analyzer, and the determined DNA sequence was compared with the GenBank database using NCBI's BLAST for identification of the strain. The process was conducted by Macrogen Inc. on behalf of the inventors of the present disclosure.

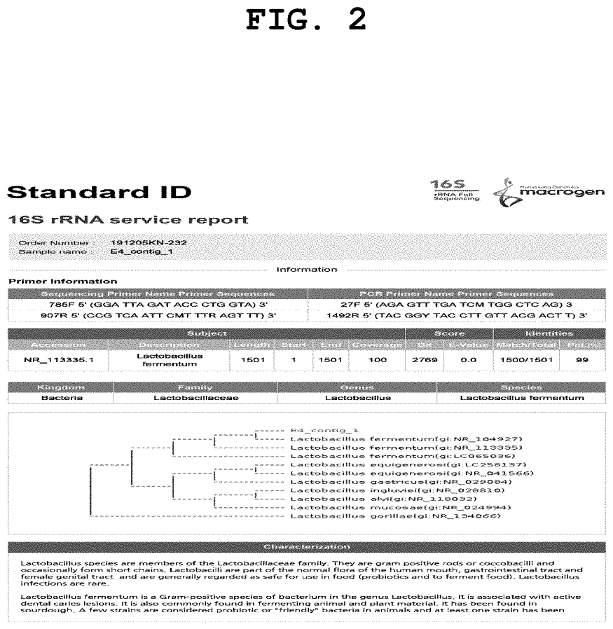

16s rRNA sequencing was performed using the strains described above. As a result, E4 and E were identified as Lactobacillus fermentum, and B and F were identified as Lactobacillus brevis. FIG. 2 shows the result of the 16S rRNA analysis of the E4 strain.

### Example 3: Establishment of Mass Production Process for Probiotic Strain

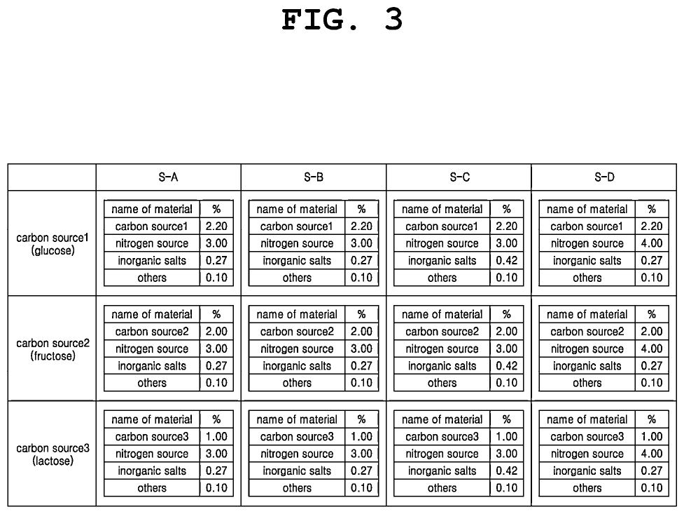

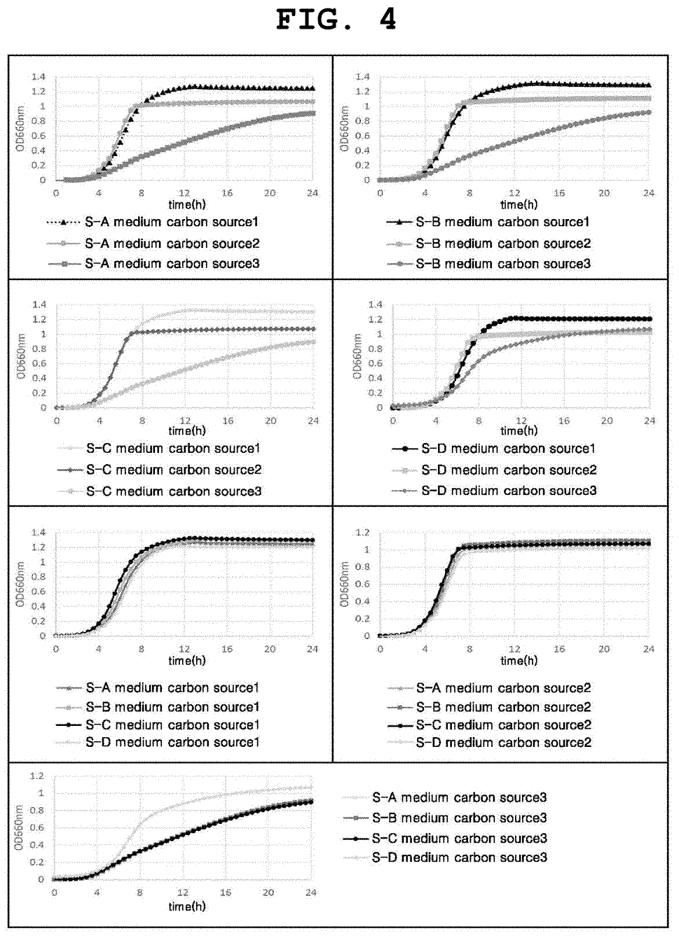

To establish a small-scale optimization production process for Lactobacillus fermentum E4 (FT E4) among the identified strains identified, a flask experiment was performed. The types and ratios of carbon and nitrogen sources and inorganic salts were varied, and the growth curves of the strains were investigated. The compositions of the media used to compare the growth rate in the media are shown in FIG. 3. FIG. 3 shows the compositions of the media used to compare the growth rates of the E4 strain. FIG. 4 shows the results of the culture of the E4 strain in various culture media.

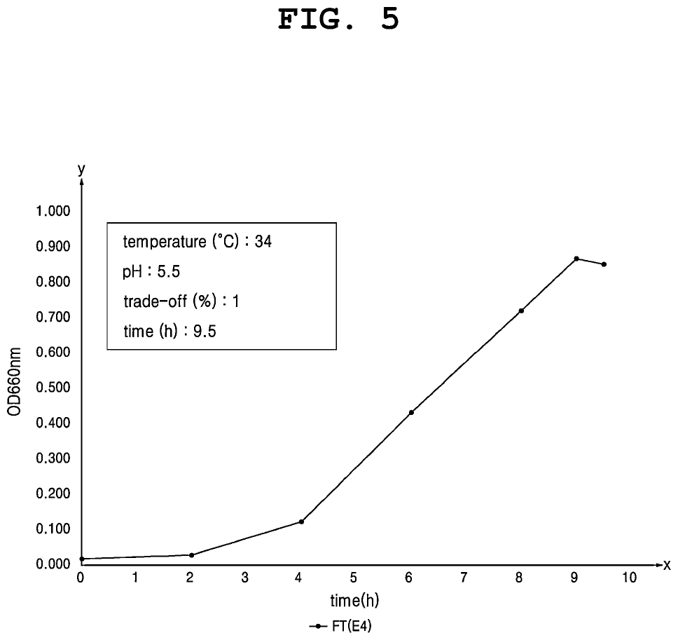

Finally, a combination of an S-C medium and Carbon Source 1 (glucose) showed the best growth rate, and it was verified through full growth, was selected as a medium of a 5 L jar fermenter and was used for mass production. The strain was cultured in a 5 L jar fermenter having an S-C medium selected as the optimal medium composition and Carbon Source 1 (glucose) for a total of 9.5 hours, and the culture medium was diluted 10 times and measured at an optical density of 660 nm. FIG. 5 is a view showing a result of a case where the E4 strain was cultured in an environment in which glucose is used as a carbon source (Carbon Source 1) and an S-C medium selected as an optimum culture medium is sued.

On the other hand, the yield of the Lactobacillus fermentum E4 strain for each process was analyzed by measuring the number of living cells in powder after the completion of the culture (OB), the mixed pellet preparation (MXPE), and the freeze-drying. The analysis results are shown in Table 4.

TABLE 4

| | OB | | | MXPE | | | | Powder | | |
|---|---|---|---|---|---|---|---|---|---|---|
| | | | Total | | | | Total | | | Total |
| | | Number | number | | | Number | number | | Number | number |
| | | of | of | | | of | of | Amount | of | of |
| | | living | living | | | living | living | of | living | living |
| | Vol | cells | cells | Pellet | MXPE | cells | cells | powder | cells | cells |
| | (ml) | (CFU/ml) | (CFU) | (g) | (g) | (CFU/g) | (CFU) | (g) | (CFU/g) | (CFU) |
| Numerical | 1400 | 1.07E+10 | 1.50E+13 | 15.87 | 31.74 | 1.17E+11 | 3.71E+12 | 6.46 | 4.35E+11 | 2.81E+12 |
| value | | | | | | | | | | |
| Yield | | | 100.00 | | | | 24.79 | | | 18.73 |
| (%) | | | | | | | | | | |

At the end of the culture, the number of living cells in the culture medium was 1.07E+10 CFU/mL, the number of living cells concentrated by centrifugation and mixed in a cryoprotectant was 1.17E+11 CFU/mL, the amount of freeze-dried powder was 6.46 g, and the number of living cells in the powder was confirmed to be 4.35E+11 CFU/g. The yield in the MXPE in the culture medium was 24.79%, and the yield in the powder was 18.73%. Based on the results of analysis using the 5 L jar fermenter, the E4 strain was determined to be a strain suitable for mass production.

Name of institution for deposit: Korea Research Institute of Bioscience and Biotechnology (KRIBB)

Accession number: KCTC14570BP

Date of deposit: May 18, 2021

What is claimed is:

1. A Lactobacillus fermentum E4 strain (KCTC 14570BP).

2. The strain according to claim 1, wherein the E4 strain is a Lactobacillus fermentum E4 strain (KCTC 14570BP) having improved antioxidant and anti-inflammatory activities.

3. An anti-inflammatory food composition comprising a Lactobacillus fermentum E4 strain (KCTC 14570BP), a culture medium for the lactobacillus fermentum E4 strain KCTC 14570BP), a concentrate of the culture medium, or a dry powder of the culture medium.

4. An anti-aging food composition comprising a Lactobacillus fermentum E4 strain (KCTC 14570BP), a culture medium for the lactobacillus fermentum E4 strain (KCTC 14570BP), a concentrate of the culture medium, or a dry powder of the culture medium.

5. A pharmaceutical composition for prevention and treatment of inflammatory diseases, the composition comprising a Lactobacillus fermentum E4 strain (KCTC 14570BP), a culture medium for the lactobacillus fermentum E4 strain (KCTC 14570BP), a concentrate of the culture medium, or a dry powder of the culture medium.

6. A method of culturing a lactobacillus fermentum E4 strain (KCTC 14570BP) in a culture medium using glucose as a carbon source.

7. The strain according to claim 6, wherein the culture medium comprises 2.00% to 2.50% (w/v) of a carbon source, 2.80% to 3.20% (w/v) of a nitrogen source, and 0.25% to 0.29% (w/v) of inorganic salts.
